(19) United States
(12) Patent Application Publication
(10) Pub. No.: US 2015/0305699 A1
(43) Pub. Date: Oct. 29, 2015

(54) RADIATION GENERATION APPARATUS

(71) Applicant: CANON KABUSHIKI KAISHA, Tokyo (JP)

(72) Inventor: Shichihei Sakuragi, Tokyo (JP)

(21) Appl. No.: 14/685,717

(22) Filed: Apr. 14, 2015

(30) Foreign Application Priority Data
Apr. 28, 2014 (JP) 2014-093167

(51) Int. Cl. A61B 6/00

(52) U.S. Cl. CPC A61B 6/4405

(57) ABSTRACT

A radiation generation apparatus includes a radiation generator configured to generate radiation, a supporting unit configured to support the radiation generator, a power supply unit configured to supply electric power to the radiation generator, and a moving unit configured to be movable with the power supply unit mounted on it. The moving unit is detachable from the supporting unit.

### BACKGROUND OF THE INVENTION

1. Field of the Invention

The present invention relates to a radiation generation apparatus including a radiation generator which generates radiation.

2. Description of the Related Art

In recent years, strong demand has arisen for a portable radiation generation apparatus excellent in portability and capable of being carried outside hospitals to make diagnoses using radiation images in home medical care, rounds in aged care facilities, and disaster sites. In a conventional radiation generation apparatus, to place importance on portability, a radiation generation source may be configured as a mono-tank structure integrally including a radiation generator which generates radiation, a controller which controls the radiation generator, an operation panel which allows an operator to input capturing conditions, and a power supply unit.

Japanese Patent Laid-Open No. 2012-29844 discloses a structure in which a plurality of medical devices having different functions are detachably attached to a moving cart having an electric power supply source.

Japanese Patent Laid-Open No. 2012-30061 discloses a structure in which a radiation generation source is detachably attached to a moving cart having an electric power supply source. A compact battery is provided in the radiation generation source. When the remaining power level of the battery becomes low, electric power is supplied to the battery from the electric power supply source of the moving cart.

A conventional mono-tank structure requires a power supply unit including a battery of a large capacity to acquire many high-quality images. In this case, the weight of the radiation generation source increases in proportion to an increase in the capacity of the power supply unit, thus impairing portability. When the radiation generation source becomes heavy, its supporting structure becomes bulky, and portability is impaired. In addition, the installation work becomes more cumbersome.

In the arrangements disclosed in Japanese Patent Laid-Open Nos. 2012-29844 and 2012-30061, portability is impaired because the moving cart including the large electric power supply source must be conveyed to the vicinity of an object subjected to capturing, together with the radiation generation source. Also, in the arrangement disclosed in Japanese Patent Laid-Open No. 2012-30061, when the radiation generation source is separated, taken out to a location where a subject exists, and captures his image in an environment where the moving cart cannot be moved in, a supporting structure for supporting the radiation generation source is required separately. This complicates the installation work of the radiation generation source.

The present invention provides a technique in which a moving unit is configured to be detachable from a supporting unit, and capturing becomes possible regardless of whether the supporting unit is arranged on the moving unit or the supporting unit is separated from the moving unit.

### SUMMARY OF THE INVENTION

According to one aspect of the present invention, there is provided a radiation generation apparatus comprising: a radiation generator configured to generate radiation; a supporting unit configured to support the radiation generator; a power supply unit configured to supply electric power to the radiation generator; and a moving unit configured to be movable with the power supply unit mounted on the moving unit, wherein the moving unit is detachable from the supporting unit.

According to another aspect of the present invention, there is provided a radiation generation apparatus comprising: a radiation generator configured to generate radiation; a supporting unit configured to support the radiation generator; a power supply unit configured to supply electric power to the radiation generator; and a moving unit including a plurality of wheels, wherein the power supply unit is detachable from the moving unit, and the moving unit is detachable from the supporting unit.

Further features of the present invention will become apparent from the following description of exemplary embodiments (with reference to the attached drawings).

### BRIEF DESCRIPTION OF THE DRAWINGS

FIG. 1 is a view showing the arrangement of a radiation generation apparatus according to the first embodiment;

FIG. 2A is a view showing an example of the layout of a radiation generator, a supporting unit, and a power supply unit;

FIG. 2B is a block diagram showing the arrangement of the power supply unit;

FIG. 3A is a view exemplifying the arrangements of the power supply unit and a moving unit;

FIG. 3B is a view showing a state in which the power supply unit is arranged on the moving unit;

FIGS. 4A and 4B are views exemplifying the arrangements of the radiation generator, supporting unit, power supply unit, and moving unit in a state at the time of conveyance;

FIG. 5 is a view showing a state in which the radiation generator and the supporting unit are separated from the moving unit;

FIG. 6 is a view showing an arrangement in which capturing is performed with a structure in which the radiation generator and the supporting unit are integrated with the moving unit;

FIGS. 7A and 7B are views showing the arrangement of a radiation generation apparatus according to the second embodiment;

FIG. 8A is a view showing the arrangement of a radiation generation apparatus according to the third embodiment;

FIG. 8B is a view showing the arrangement of the radiation generation apparatus according to the third embodiment;

FIGS. 9A and 9B are views showing the arrangement of a radiation generation apparatus according to the fourth embodiment;

FIG. 10 is a view showing the arrangement of a radiation generation apparatus according to the fifth embodiment;

FIGS. 11A, 11B, and 11C are a view and block diagrams, respectively, showing the arrangement of a radiation generation apparatus according to the sixth embodiment;

FIGS. 12A and 12B are views showing the arrangement of a radiation generation apparatus according to the seventh embodiment;

FIGS. 13A and 13B are a block diagram and a view, respectively, showing the arrangement of the radiation generation apparatus according to the seventh embodiment; and

FIGS. 14A, 14B, 14C, and 14D are views showing the arrangement of the radiation generation apparatus according to the seventh embodiment.

### DESCRIPTION OF THE EMBODIMENTS

Embodiments of the present invention will be exemplarily described in detail below with reference to the accompanying drawings. Note that the constituent elements described in the embodiments are merely examples. The technical scope of the present invention is determined by the scope of claims and is not limited by the following individual embodiments.

### First Embodiment

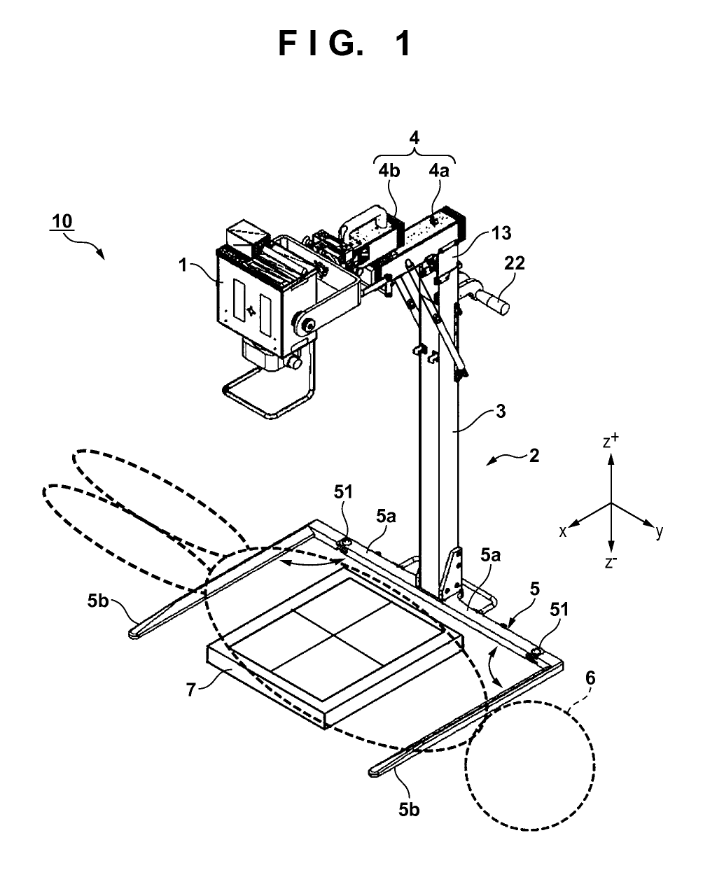

FIG. 1 is a view showing the arrangement of a portable radiation generation apparatus (to be simply referred to as a radiation generation apparatus hereinafter) according to the first embodiment. The radiation generation apparatus according to this embodiment includes a radiation generator 1 which generates radiation, a supporting unit 2 which supports the radiation generator 1, a power supply unit 9 which supplies electric power to the radiation generator 1, and a moving unit 8 capable of moving with the power supply unit 9 mounted on it. In this case, the moving unit 8 (cart) is detachable from the supporting unit 2. The moving unit includes a holding member 17 which detachably holds the supporting unit 2. The supporting unit 2 has a self-standing mechanism in which the supporting unit 2 supports the radiation generator 1 and stands by itself. The supporting unit 2 integrated with the radiation generator 1 includes an engaging member which detachably engages with the moving unit 8 (cart).

The supporting unit 2 includes a boom 4 which supports the radiation generator 1 in the horizontal direction (x direction in FIG. 1), a column 3 which supports the boom 4 in the vertical direction (z direction in FIG. 1), and a supporting leg 5 which supports the column 3.

To ensure portability, the supporting unit 2 includes a folding mechanism (rotation unit 13) which supports the boom 4 so that the boom 4 can rotate. FIG. 1 shows a state at the time of capturing in which the boom 4 supports the radiation generator 1 in the horizontal direction. When the state at the time of capturing shifts to a state at the time of conveying/accommodating the apparatus, the rotation unit 13 rotates the boom 4 supported in the horizontal direction toward a vertically down position (z-direction in FIG. 1). The rotated boom 4 supports the radiation generator 1 at the vertically down position. With the arrangement in which the boom 4 is supported to be rotatable, the heavy radiation generator 1 is supported at the vertically down position at the time of conveyance/accommodation. This can make the overall apparatus compact and improve portability.

In shift from the state at the time of conveyance/accommodation to the state at the time of capturing, the rotation unit 13 rotates, in the horizontal direction (x direction in FIG. 1), the boom 4 supported at the vertically down position, and the rotated boom 4 supports the radiation generator 1 in the horizontal direction. Although the supporting unit 2 is connected to the radiation generator 1, the radiation generator 1 and the supporting unit 2 may have a detachable structure in consideration of assembly and maintenance.

The boom 4 includes a stationary boom 4a and a movable boom 4b. One end of the movable boom 4b is supported by one end of the stationary boom 4a so that it can move (expand and contract) in the horizontal direction (x direction in FIG. 1) via a sliding member (for example, a guide rail or roller). The radiation generator 1 is connected to the other end side of the movable boom 4b. The other end side of the stationary boom 4a is supported at the top of the column 3 via the folding mechanism (rotation unit 13). By constituting the boom 4 so that it can expand and contract, the overall apparatus can be made compact at the time of conveyance/accommodation, thereby improving portability.

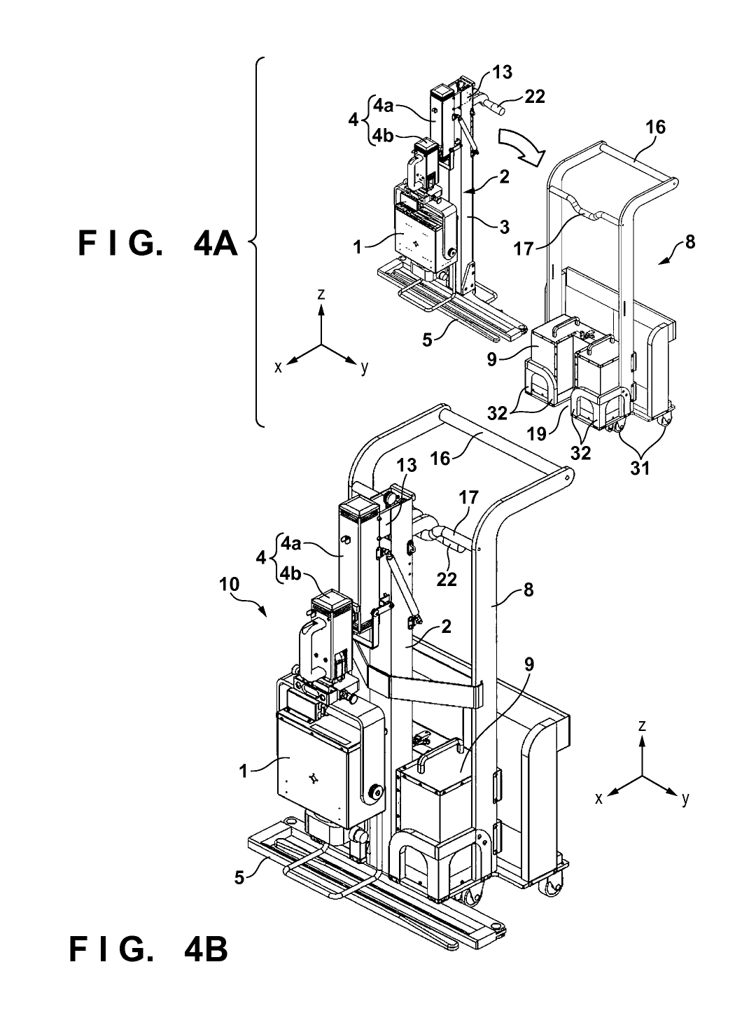

The supporting leg 5 is connected to the lower end of the column 3, and supports the radiation generator 1, the boom 4, and the column 3. The supporting leg 5 includes stationary legs 5a fixed to the column 3, movable legs 5b which move with respect to the stationary legs 5a, and rotation members 51 which support the movable legs 5b to be rotatable with respect to the stationary legs 5a. In a state in which the moving unit 8 (cart) holds the supporting unit 2, the movable legs 5b are folded with respect to the stationary legs 5a by rotation of the rotation members (FIGS. 4A and 4B). In a state in which the supporting unit 2 is separated from the moving unit, the movable legs 5b are extended with respect to the stationary legs 5a by rotation of the rotation members (FIGS. 1, 2A, and 5).

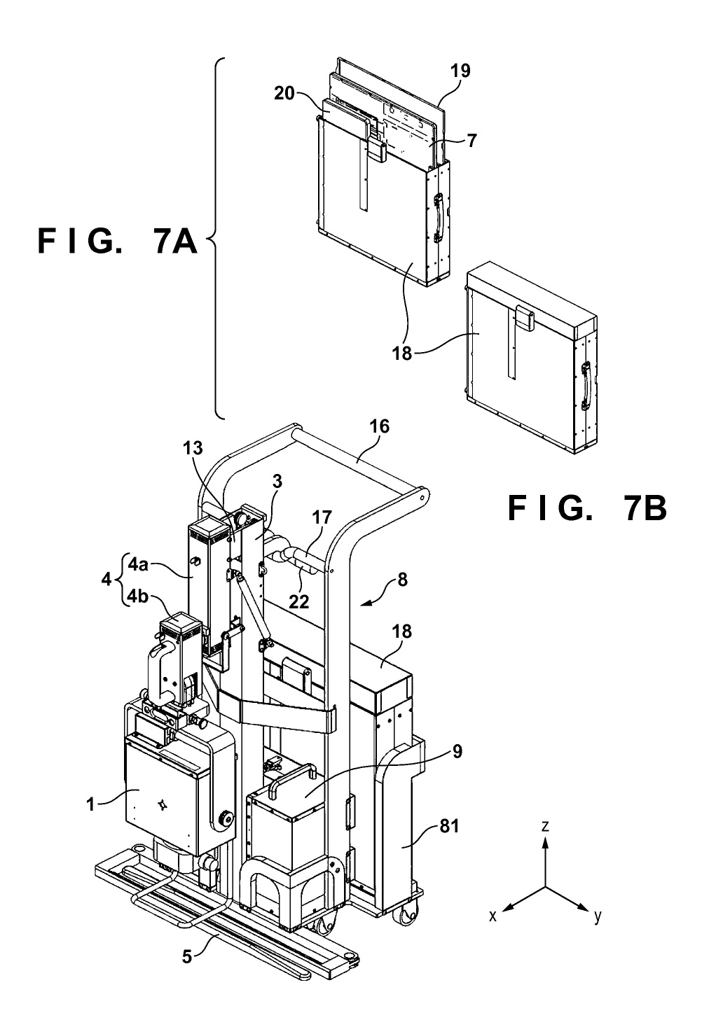

A radiation detector 7 is arranged immediately below an object 6. The radiation detector 7 detects radiation which has irradiated the object 6 from the radiation generator 1 and has passed through the object 6. Then, the radiation detector 7 converts the radiation into an image based on the radiation detection result. The image acquired by the radiation detector 7 is transmitted to and processed by an information processing apparatus 20 (FIG. 7A).

The distance between the radiation detector 7 and the radiation generator 1 in the vertical direction in a state in which the boom 4 is held at a position in the horizontal direction is designed to ensure a predetermined capturing distance. This obviates the need to adjust the capturing distance after the object 6 is arranged immediately below the radiation generator 1 when performing capturing.

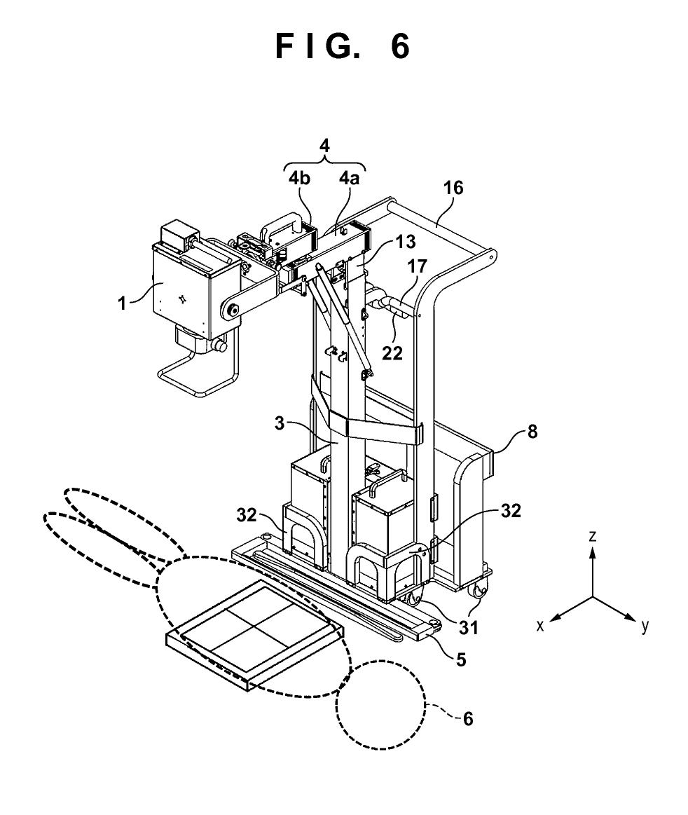

When capturing is performed in a state in which the supporting unit 2 is held on the moving unit 8 (cart) by the holding member 17, the radiation generator 1 generates radiation on the moving unit (FIG. 6). When capturing is performed in a state in which holding by the holding member 17 is released and the supporting unit 2 is separated from the moving unit 8 (cart), the radiation generator 1 generates radiation in a state in which the supporting unit 2 supports the radiation generator 1 (FIGS. 1, 2A, and 5).

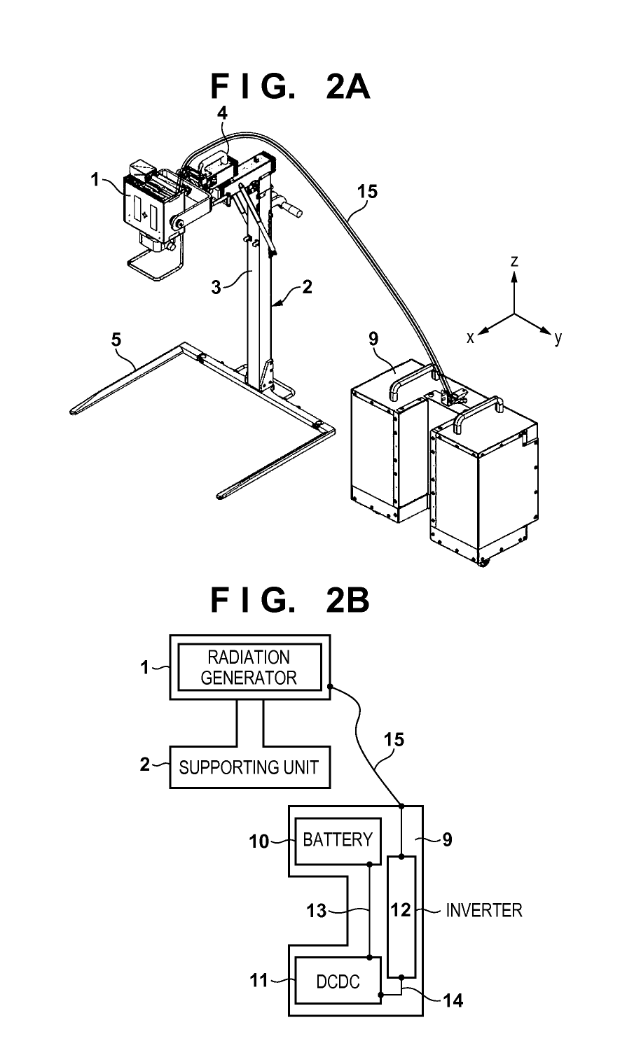

When capturing is performed in the state in which the supporting unit 2 is separated from the moving unit 8 (cart), the supporting unit 2 needs to be reduced in size and weight in order to improve the efficiency of the installation work of the apparatus. To achieve this, the radiation generator 1 also needs to be reduced in size and weight. FIG. 2A is a view showing an example of the layout of the radiation generator 1, the supporting unit 2 (boom 4, column 3, and supporting leg 5), and the power supply unit 9. FIG. 2B is a block diagram showing the arrangement of the power supply unit. In the radiation generation apparatus according to this embodiment, the power supply unit 9 which supplies electric power is separated as another structure from the radiation generator 1 in order to reduce the size and weight of the radiation generator 1.

As shown in FIG. 2B, a battery 10 for supplying electric power to the radiation generator 1, a DC-DC converter 11 for boosting the voltage of electric power, and an inverter 12 for converting an electric power waveform are arranged inside the power supply unit 9.

Electric power accumulated in the battery 10 is supplied to the radiation generator 1 at the time of capturing. When electric power enough to ensure high image quality is supplied to the radiation generator 1 via a cable 15, it is necessary to raise the voltage and decrease the current in order to suppress heat generation of the cable 15. For this purpose, electric power supplied from the battery 10 inside the power supply unit 9 is input to the DC-DC converter 11 via a cable 13 to raise the voltage of supply electric power. Then, the boosted electric power is input from the DC-DC converter 11 to the inverter 12 via a cable 14. The inverter 12 converts the supplied electric power into a waveform necessary for capturing, and inputs the converted electric power to the radiation generator 1 via the cable 15.

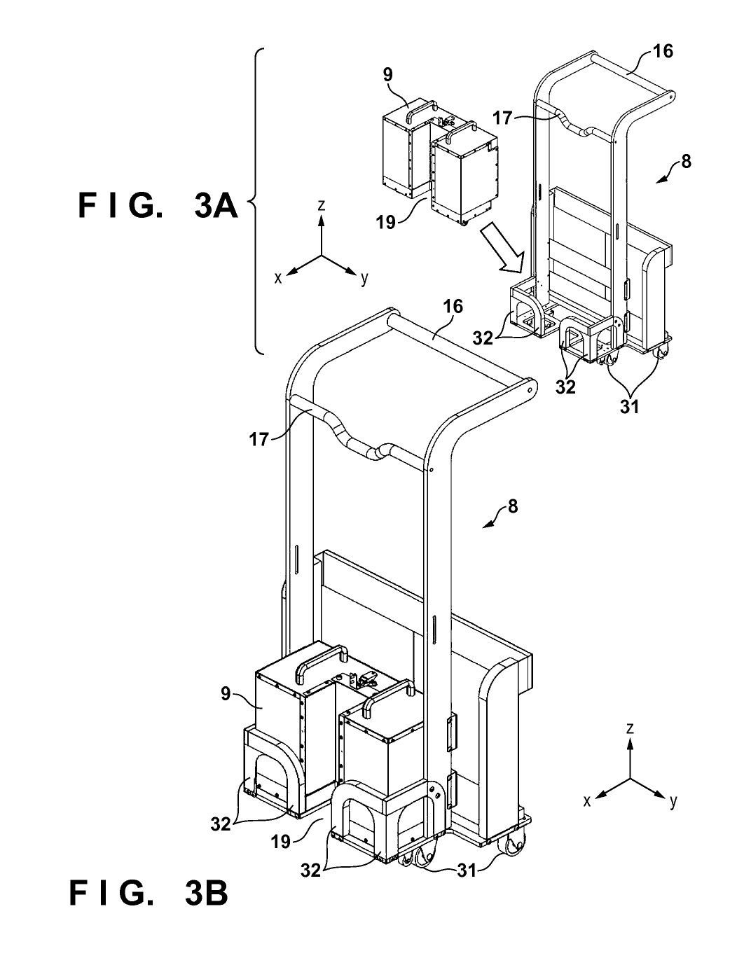

FIG. 3A is a view exemplifying the arrangements of the power supply unit 9 and moving unit 8 (cart). FIG. 3B is a view showing a state in which the power supply unit 9 is arranged on the moving unit (cart). The power supply unit 9 having the arrangement as described with reference to FIGS. 2A and 2B is heavy. To improve portability, the radiation generation apparatus according to this embodiment includes the moving unit 8 (cart) for conveying the power supply unit 9.

As shown in FIGS. 3A and 3B, the moving unit 8 (cart) includes wheels 31 for simplifying the conveyance work, and a handle 16 to be operated by the operator. The moving unit 8 (cart) also includes power supply holding members 32 which detachably hold the power supply unit 9. The power supply unit 9 is detachable from the moving unit 8 (cart). When the power supply unit 9 is arranged on the moving unit 8 (cart), the power supply holding members 32 constrain the movement of the power supply unit 9 in the back-and-forth direction (x direction) and the right-and-left direction (y direction), and the power supply unit 9 is held on the moving unit 8 (cart).

FIG. 4A is a view exemplifying the arrangements of the radiation generator 1, supporting unit 2 (boom 4, column 3, and supporting leg 5), power supply unit 9, and moving unit 8 (cart) in a state at the time of conveyance/accommodation. FIG. 4B is a view showing a state in which the radiation generator 1, supporting unit 2 (boom 4, column 3, and supporting leg 5), and power supply unit 9 are arranged on the moving unit 8 (cart). The moving unit 8 (cart) includes the holding member 17 which detachably holds the supporting unit 2 integrated with the radiation generator 1. The supporting unit 2 includes an engaging member 22 projecting from the column 3. A step (concave portion) is formed in the holding member 17 of the moving unit 8 (cart). The engaging member 22 of the supporting unit 2 is fitted in the step (concave portion) of the holding member 17, and the supporting unit 2 is held by the moving unit 8 (cart).

When arranging the radiation generator 1 and the supporting unit 2 (boom 4, column 3, and supporting leg 5) on the moving unit 8 (cart) in a state in which the power supply unit 9 is held on the moving unit 8 (cart), as shown in FIG. 4B, the engaging member 22 of the supporting unit 2 engages with the holding member 17 of the moving unit 8 (cart), and the supporting unit 2 is held on the moving unit 8 (cart). At this time, the radiation generator 1, the supporting unit 2, the power supply unit 9, and the moving unit 8 (cart) are integrated into a portable structure. Note that the holding member 17 can also be constituted to hold the supporting unit 2 by fastening using a screw or suction using a magnet or sucker, other than the structure in which the engaging member 22 of the supporting unit 2 is hooked and engaged. In the radiation generation apparatus according to this embodiment, the radiation generator 1, the supporting unit 2, and the power supply unit 9 can be arranged as an integral cart structure on the moving unit 8 (cart) at the time of conveyance. The operator can easily convey the radiation generator 1, the supporting unit 2, and the power supply unit 9.

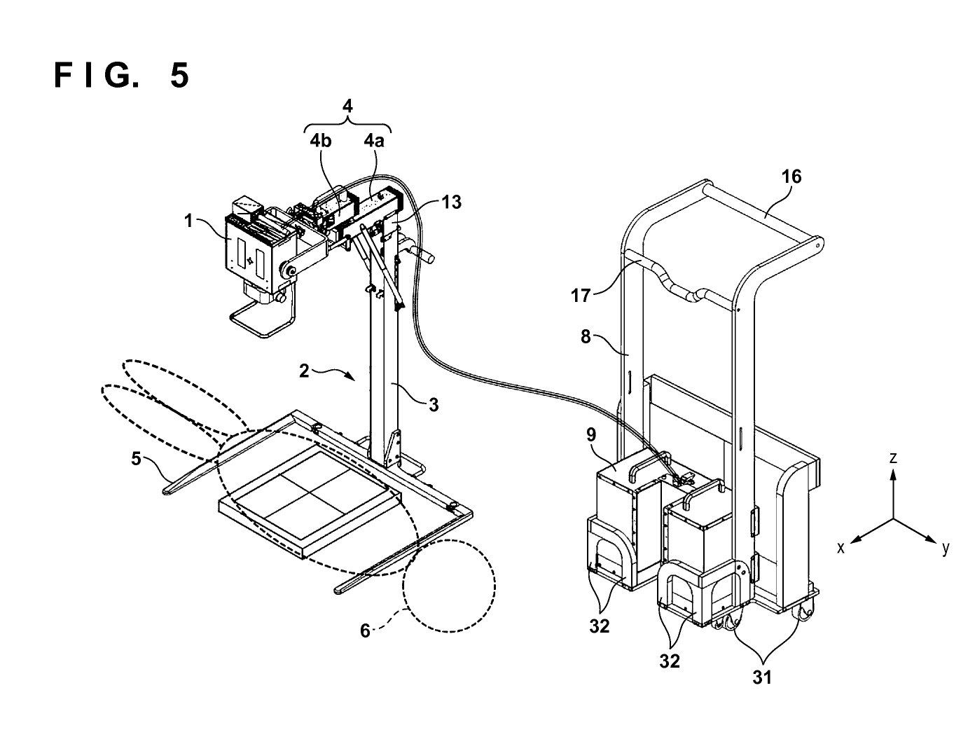

FIG. 5 is a view showing a state in which the radiation generator 1 and the supporting unit 2 are separated from the moving unit 8 (cart). At the time of conveyance, the radiation generator 1, the supporting unit 2, and the power supply unit 9 are arranged as an integral cart structure on the moving unit 8 (cart), as shown in FIG. 4B. At the time of capturing, the radiation generator 1 and the supporting unit 2 are separated from the moving unit 8 (cart) and the power supply unit 9, as shown in FIG. 5. Then, the radiation generator 1 and the supporting unit 2 are set on a bed on which the object 6 lies. The folded supporting leg 5 is extended, the boom 4 is raised, and the radiation generator 1 is held at the position in the horizontal direction. By only this installation work, a state in which capturing is possible is set.

Note that when the object 6 lies on a bed (on the same plane as that of the moving unit 8) near the moving unit 8 (cart), for example, the separation work of the radiation generator 1 and supporting unit 2 becomes unnecessary, as shown in FIG. 6. In this case, capturing can also be performed with an unchanged structure in which the radiation generator 1 and the supporting unit 2 are integrated with the moving unit 8 (cart). In this case, only by raising the boom 4 and holding the radiation generator 1 at the position in the horizontal direction without extending the supporting leg 5, the installation work can be further simplified.

### Second Embodiment

The second embodiment will further explain an arrangement in which accessories used for capturing by a radiation generation apparatus are conveyed together with a moving unit 8, in addition to the arrangement, described in the first embodiment, in which a radiation generator 1, a supporting unit 2, and a power supply unit 9 are arranged as an integral cart structure on the moving unit 8 (cart).

The same reference numerals as those in FIGS. 7A and 7B to be referred to denote the same parts, and a repetitive description thereof will be omitted. FIGS. 7A and 7B are views showing the arrangement of a radiation generation apparatus according to the second embodiment. The moving unit 8 (cart) further includes an accommodation unit 18 (conveyance box) for moving accessories together with the moving unit 8 (cart). The accessories include a radiation detector 7 which detects radiation, a grid 19 which is used together with the radiation detector 7 at the time of capturing and removes the scattered rays of radiation, and an information processing apparatus 20 capable of processing an image based on a capturing operation and the detection result of the radiation detector 7. The accommodation unit 18 (conveyance box) has a structure which allows the operator to take out these contents at the time of capturing.

The moving unit 8 (cart) includes an accommodation holding member 81 which holds the accommodation unit 18 (conveyance box). When the accommodation unit 18 is arranged on the moving unit 8 (cart), the accommodation holding member 81 constrains the movement of the accommodation unit 18 in the back-and-forth direction (x direction) and the right-and-left direction (y direction), and the accommodation unit 18 is held on the moving unit 8 (cart). The accommodation unit 18 is not constrained in the vertical direction (z direction) and is detachable from the moving unit 8 (cart).

By mounting, on the moving unit 8, the accommodation unit 18 which accommodates the radiation detector 7, the grid 19, and the information processing apparatus 20, building components necessary for radiation capturing can be conveyed by the moving unit 8 (cart). The radiation generation apparatus excellent in workability at the time of conveyance can be provided.

### Third Embodiment

The third embodiment will further explain an overturning prevention arrangement aiming to stabilize the apparatus balance at the time of conveyance/capturing, in addition to the arrangement, described in the first and second embodiments, in which a radiation generator 1, a supporting unit 2, a power supply unit 9, and an accommodation unit 18 are arranged as an integral cart structure on a moving unit 8.

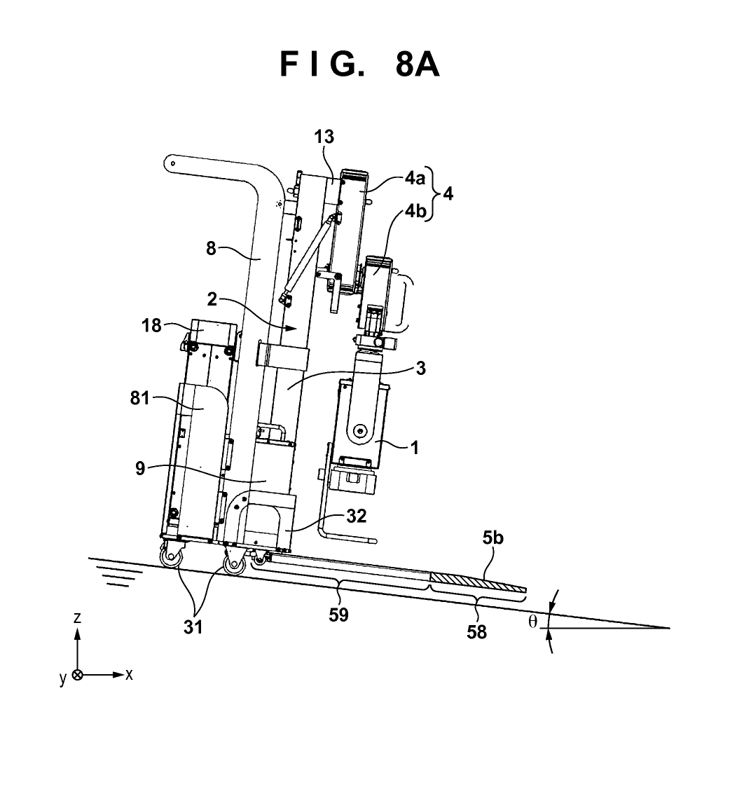

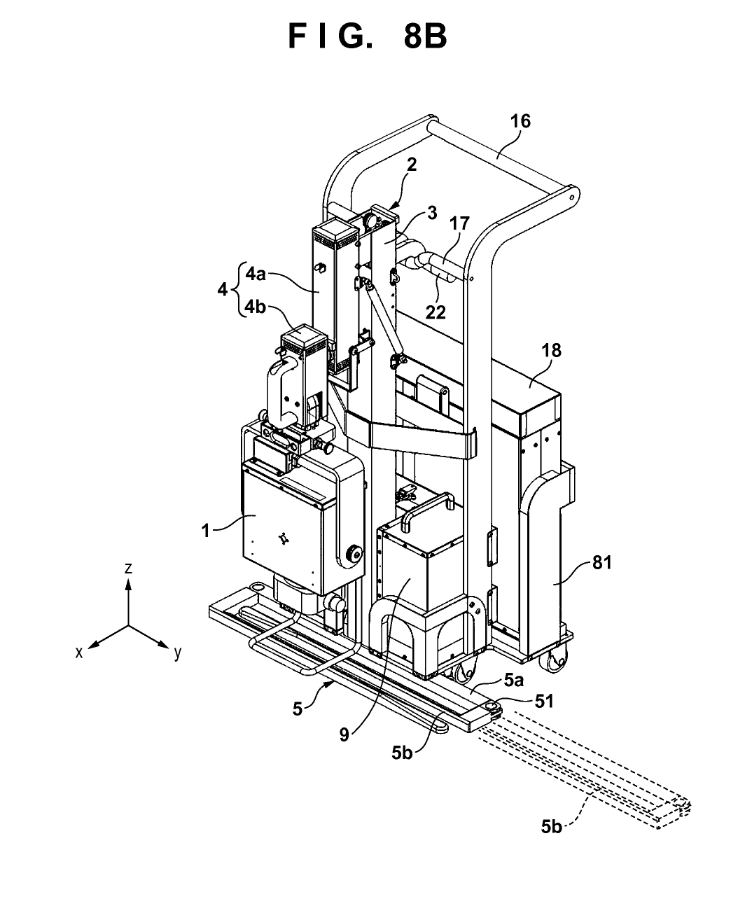

FIG. 8A is a view showing the arrangement of a radiation generation apparatus according to the third embodiment. In an example shown in FIG. 8A, the installation surface of the moving unit 8 is inclined by an inclined angle θ with respect to the horizontal. In outdoor capturing at, for example, a disaster site where the radiation generation apparatus is used, conveyance and capturing on an irregular ground or slope where the installation surface of the moving unit 8 is not horizontal are presumed. Since the radiation generator 1 which acquires a high-quality image tends to be heavy, the barycentric balance of the apparatus may be lost at a site where the horizontal is not guaranteed at the time of conveyance/capturing on an irregular ground or slope.

To stabilize the apparatus balance at the time of conveyance/capturing, the radiation generation apparatus according to this embodiment prevents forward overturning of the apparatus on a slope by extending a supporting leg 5 of the supporting unit 2 forward (x direction), as shown in FIG. 8A, from the state at the time of conveyance shown in FIG. 4B.

As described with reference to FIG. 1, the supporting leg 5 includes stationary legs 5a fixed to a column 3, movable legs 5b which move with respect to the stationary legs 5a, and rotation members 51 which support the movable legs 5b to be rotatable with respect to the stationary legs 5a. The movable legs 5b are configured to be extensible from the stationary legs 5a.

In a state in which the installation surface of the moving unit 8 is inclined by the inclined angle θ from the horizontal, the rotation members 51 rotate by the gravity acting in accordance with the inclined angle θ of the installation surface, and the movable legs 5b change from a state (for example, FIG. 4B) in which they are folded with respect to the stationary legs 5a to a state (FIG. 8A) in which they are extended.

As the arrangement in which the movable legs 5b of the supporting leg 5 are extended, the specific gravity of distal ends 58 of the movable legs 5b is set to be higher than that of other portions 59. The movable legs 5b can therefore be configured to automatically extend by the action of gravity in accordance with the inclination of the conveyance/installation surface.

The members of the distal ends 58 of the movable legs 5b and those of the other portions 59 are constituted by members different in specific gravity, and the members of the distal ends 58 are constituted by members higher in specific gravity than those of the other portions 59. Hence, the specific gravity of the members of the distal ends 58 of the movable legs 5b can also be changed. The arrangement in which the specific gravity of the members of the distal ends 58 of the movable legs 5b is changed is not limited to this example, and weights may be added to the distal ends 58 of the movable legs 5b.

Note that the overturning prevention arrangement aiming to stabilize the apparatus balance at the time of conveyance/capturing is not limited to the arrangement in which the movable legs 5b are extended in the x direction, as shown in FIG. 8A, and can also be an arrangement in which the supporting leg 5 is extended in the y direction, as shown in FIG. 8B. For example, assume that the supporting leg 5 is constituted by the stationary legs 5a and the movable legs 5b, one end of each movable leg 5b is supported by one end of the corresponding stationary leg 5a so that it can move (expand and contract) in the right-and-left direction (y direction in FIG. 1) via a sliding member (for example, a guide rail or roller).

When the conveyance/installation surface is inclined in the y direction, the radiation generation apparatus according to this embodiment extends the movable legs 5b so as to project laterally (y direction), as shown in FIG. 8B, from the state at the time of conveyance shown in FIG. 4B. This arrangement can prevent the apparatus from overturning laterally on a slope. As the arrangement in which the movable legs 5b are extended laterally, the movable legs 5b can be configured to automatically extend by the movement of the sliding member caused by gravity acting in accordance with the inclination of the conveyance/installation surface, by setting the specific gravity of the movable legs 5b to be higher than that of the stationary legs 5a. Also, the specific gravity of the movable legs 5b with respect to the stationary legs 5a can be changed by constituting the movable legs 5b and the stationary legs 5a by members different in specific gravity, and constituting the movable legs 5b by members higher in specific gravity than the stationary legs 5a.

According to the third embodiment, the apparatus balance at the time of conveyance/capturing can be stabilized.

### Fourth Embodiment

The fourth embodiment will explain an arrangement in which a shielding member against the radiation is arranged, in addition to the arrangement, described in the first and second embodiments, in which a radiation generator 1, a supporting unit 2, a power supply unit 9, and an accommodation unit 18 are arranged as an integral cart structure on a moving unit 8.

In order to prevent exposure to radiation at the time of capturing, the operator who performs radiation capturing needs to move apart from a radiation generation apparatus by a distance enough to decrease the intensity of the radiation, or install a shielding member, hide behind it, and then perform capturing. The shielding member installation work is a factor of decreasing the work efficiency at the time of capturing.

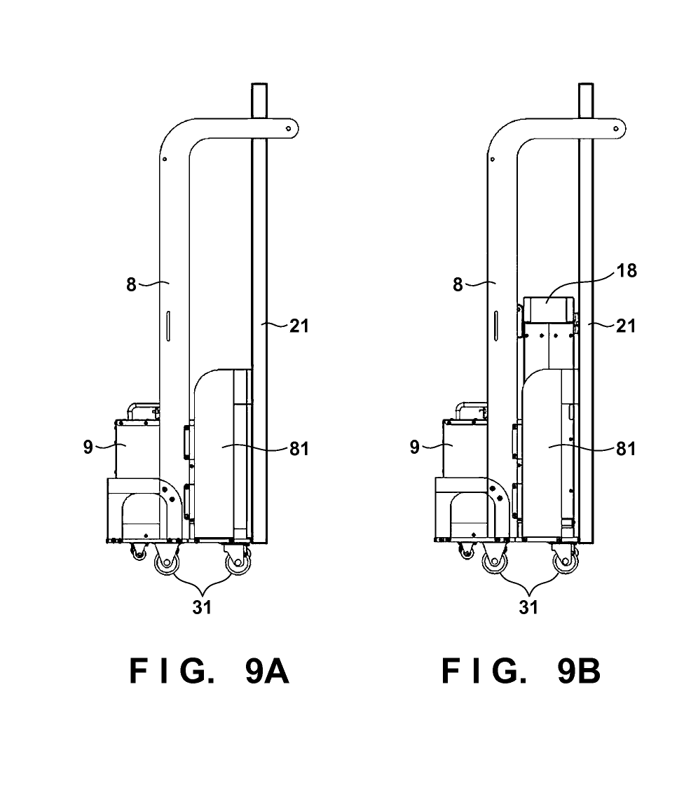

An arrangement will be explained, in which a shielding member 21 is arranged in a radiation generation apparatus according to this embodiment. FIGS. 9A and 9B are views showing the arrangement of the radiation generation apparatus according to the fourth embodiment. FIG. 9A shows an arrangement in which the shielding member 21 against the radiation is arranged on the moving unit 8 (cart). FIG. 9B shows an arrangement in which the shielding member 21 is arranged in the accommodation unit 18. According to the radiation generation apparatus of this embodiment, the shielding member 21 can be conveyed by the moving unit 8 (cart) together with the radiation generator 1, the supporting unit 2, and the power supply unit 9.

Therefore, the operator can perform capturing without moving apart from the radiation generation apparatus by a distance enough to decrease the intensity of the radiation. Alternatively, the operator can hide behind the shielding member 21 of the moving unit 8 (cart) or accommodation unit 18 and then perform capturing without separately installing a shielding member. Capturing can be performed without decreasing the work efficiency.

### Fifth Embodiment

The fifth embodiment will explain the detailed arrangement of an engaging member 22 in the arrangement, described in the first embodiment, in which a radiation generator 1, a supporting unit 2, and a power supply unit 9 are arranged as an integral cart structure on a moving unit 8 (cart). Note that the engaging member 22 can also function as a handle used when the operator performs the apparatus installation work.

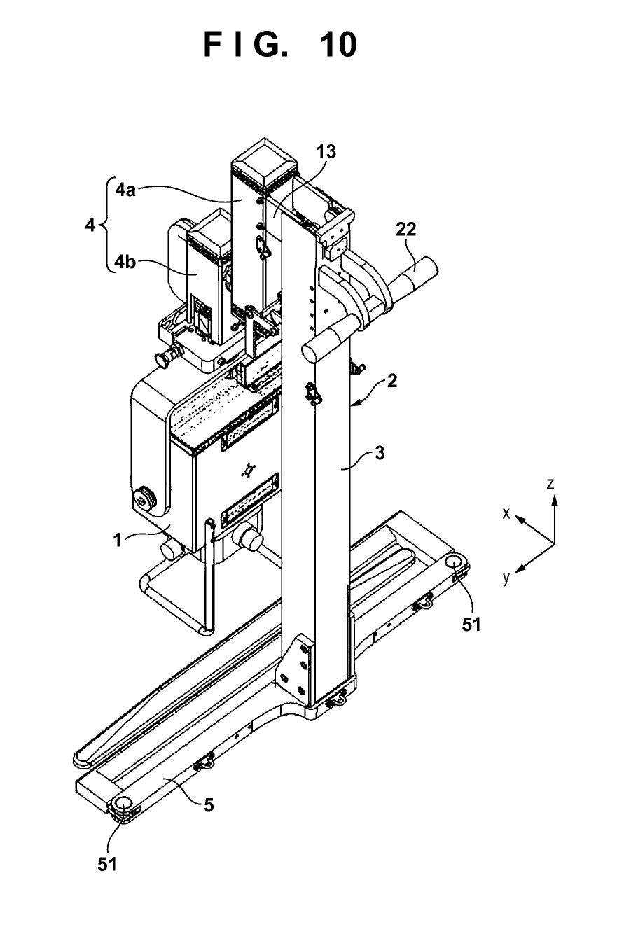

FIG. 10 is a view showing the arrangement of a radiation generation apparatus according to the fifth embodiment. The handle (engaging member 22) is attached to a column 3. The handle (engaging member 22) is attached so that the operator can efficiently perform the apparatus installation work. The handle (engaging member 22) is arranged on the center line of the column 3 so as not to lose the barycentric balance of the apparatus at the time of work. By engaging the engaging member 22 with a holding member 17 of the moving unit 8 (cart), the moving unit 8 (cart) can hold the supporting unit 2. At this time, the radiation generator 1, the supporting unit 2, the power supply unit 9, and the moving unit 8 (cart) are integrated into a conveyable structure.

Note that a magnet or sucker may be attached to the handle (engaging member 22) to fix the supporting unit 2 to the moving unit 8 (cart) by using the magnetic force or suction force. It is also possible to form a screw portion on the handle (engaging member 22), form a nut portion on the moving unit 8 (cart), and fix the supporting unit 2 to the moving unit 8 (cart) by fastening of the screw portion.

The radiation generation apparatus according to the fifth embodiment allows the operator to efficiently perform the apparatus installation work via the handle (engaging member 22). At the time of conveying the apparatus, the radiation generator 1, the supporting unit 2, and the power supply unit 9 can be arranged as an integral cart structure on the moving unit 8 by using the handle (engaging member 22). The operator can easily convey the radiation generator 1, the supporting unit 2, and the power supply unit 9.

### Sixth Embodiment

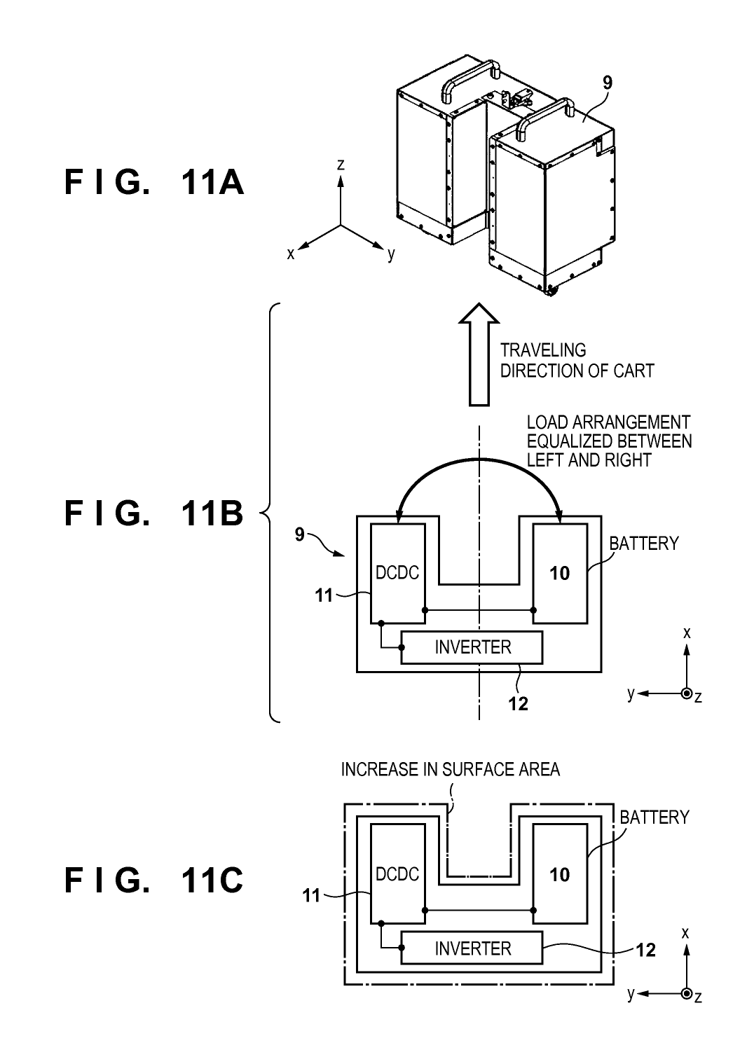

The sixth embodiment will further explain in detail an example of the arrangement of a power supply unit 9, in addition to the arrangement, described in the first embodiment, in which a radiation generator 1, a supporting unit 2, and the power supply unit 9 are arranged as an integral cart structure on a moving unit 8 (cart). FIGS. 11A, 11B, and 11C are a view and block diagrams, respectively, showing the arrangement of a radiation generation apparatus according to the sixth embodiment. In a form in which the power supply unit 9 is mounted on the moving unit 8 (cart), if the load is localized to a wheel on either the left or right side in the traveling direction, the moving unit 8 tends to travel in a direction in which the load is heavier. The operator may feel it difficult to move the moving unit 8 in a direction intended by him.

A structure in which the load is uniformly applied to the left and right wheels in the traveling direction of the moving unit 8 (cart) improves the operability of the operator. The building components of the power supply unit 9 are arranged with a weight distribution equalized between left and right in the traveling direction of the moving unit 8 (cart). More specifically, as shown in FIGS. 11A, 11B, and 11C, the power supply unit 9 has an arrangement in which a battery 10 and DC-DC converter 11, which are heavy among building components, are arranged left and right in the traveling direction of the moving unit 8 (cart). The power supply unit 9 also has an arrangement in which an inverter 12 lighter than the battery 10 and the DC-DC converter 11 is arranged at the center. Note that the layout of the battery 10 and DC-DC converter 11 is not limited to the example shown in FIGS. 11B and 11C. It is also possible to arrange the battery 10 on the left side on the paper surface, and arrange the DC-DC converter 11 on the right side on the paper surface. Even in this case, the building components of the power supply unit 9 can be arranged with a weight distribution equalized between left and right in the traveling direction of the moving unit 8 (cart).

In this manner, the building components of the power supply unit 9 are arranged so that loads become equal between the left and right sides in the traveling direction. This arrangement can solve the problem that the moving unit 8 tends to move toward a heavier-load side owing to the localization of the load. The operator can move forward the moving unit 8 (cart) in a direction intended by him, and the operability can be improved.

Since the power supply unit 9 has a fear of temperature rise caused by heat generation, it is desired to increase the surface area in order to improve the heat radiation efficiency. To do this, as shown in FIGS. 11A, 11B, and 11C, a recess (concave portion) is formed in the outer shape of the housing along the arrangement of the building components inside the power supply unit 9. This increases the surface area from which heat from the building components is radiated, and improves the heat radiation efficiency.

The arrangement of the power supply unit 9 according to the sixth embodiment can improve the operability of the moving unit 8 (cart). In addition, the power supply unit excellent in heat radiation efficiency can be provided.

### Seventh Embodiment

The seventh embodiment will further explain in detail a method of charging a battery 10 of a power supply unit 9, in addition to the arrangement, described in the first embodiment, in which a radiation generator 1, a supporting unit 2, and the power supply unit 9 are arranged as an integral cart structure on a moving unit 8 (cart). A radiation generation apparatus includes the radiation generator 1 which generates radiation, the supporting unit 2 which supports the radiation generator 1, the power supply unit 9 which supplies electric power to the radiation generator 1, and the moving unit 8 having a plurality of wheels 31. The power supply unit 9 is configured to be detachable from the moving unit 8. The moving unit 8 is configured to be detachable from the supporting unit 2.

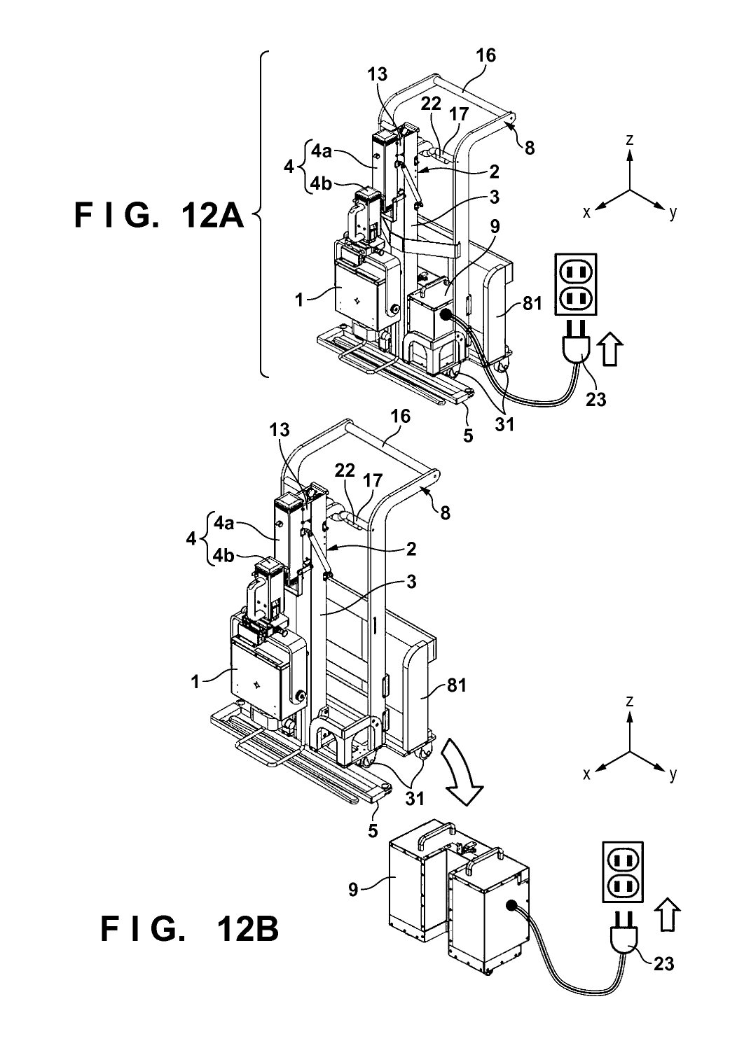

FIGS. 12A and 12B to FIGS. 14A to 14D are views for explaining the arrangement of the radiation generation apparatus according to the seventh embodiment. FIGS. 12A and 12B are views exemplifying an arrangement for charging the battery of the power supply unit. As shown in FIGS. 12A and 12B, the power supply unit 9 includes an AC plug 23 connectable to an AC power supply so that the battery running out of electric power can be charged. Assume that the AC plug 23 is electrically connected to the battery 10 inside the power supply unit 9, and electric power supplied from the AC plug 23 charges the battery 10 of the power supply unit 9.

The battery 10 of the power supply unit 9 can be charged even in a conveyance form in which the supporting unit 2 integrated with the radiation generator 1 is connected to the moving unit 8 (cart) on which the power supply unit 9 is mounted. Since the power supply unit 9 has a structure detachable from the moving unit 8 (cart), it is also possible that the power supply unit 9 is detached from the moving unit 8 (cart), the single power supply unit 9 is conveyed to a charging location, and the battery 10 is charged through the AC plug 23.

FIG. 13A is a plan view showing the arrangement of the building components of the power supply unit 9. FIG. 13A shows an example in which the battery 10 is arranged on the left side on the paper surface, and a DC-DC converter 11 is arranged on the right side on the paper surface. FIG. 13B is a view showing an example in which the battery 10 is configured to be detachable from the power supply unit 9.

To reduce the work burden when the heavy power supply unit 9 is detached and conveyed at the time of charging, the power supply unit 9 according to this embodiment is configured so that only the battery 10 is detachable, out of the building components of the power supply unit 9. The AC plug 23 is attached to the battery 10. At the time of charging, only the battery can be detached from the housing of the power supply unit 9 and conveyed to charge the battery 10 by electric power supplied through the AC plug 23. Since the battery 10 is configured to be detachable from the housing of the power supply unit 9, only the battery can be conveyed and charged at the time of charging, thereby reducing the burden on the operator. Further, a standby battery can also be used in addition to the battery 10. The standby battery is charged in advance and attached to the power supply unit 9 during charging of the battery 10. This makes it possible to perform capturing work using the standby battery before the completion of charging the battery 10.

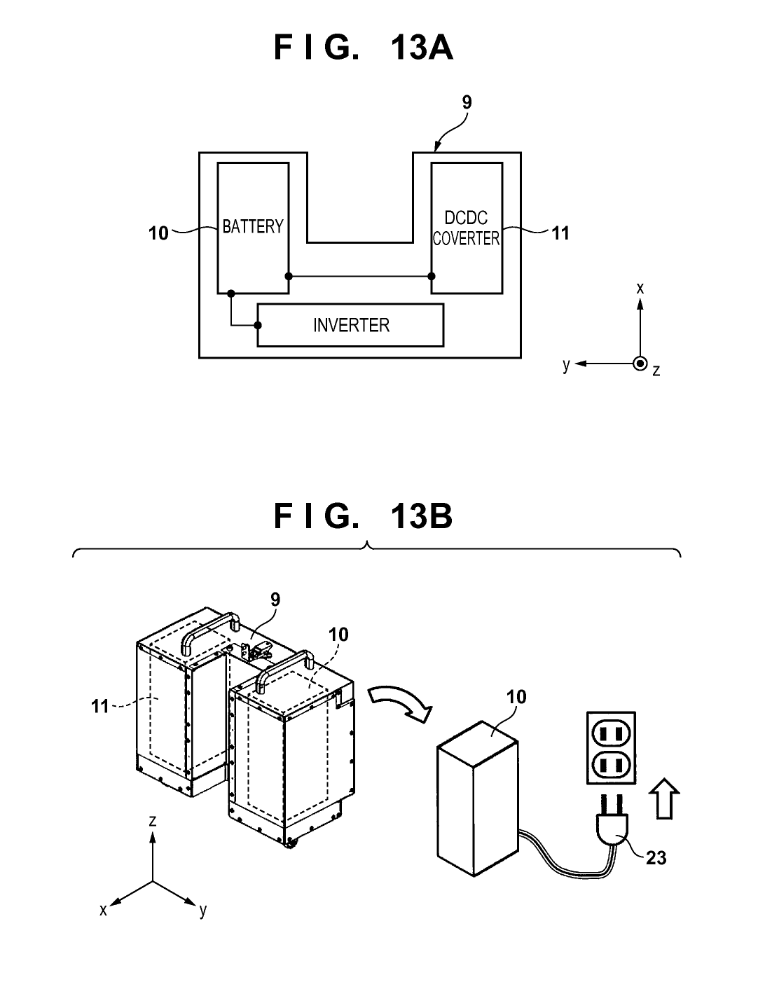

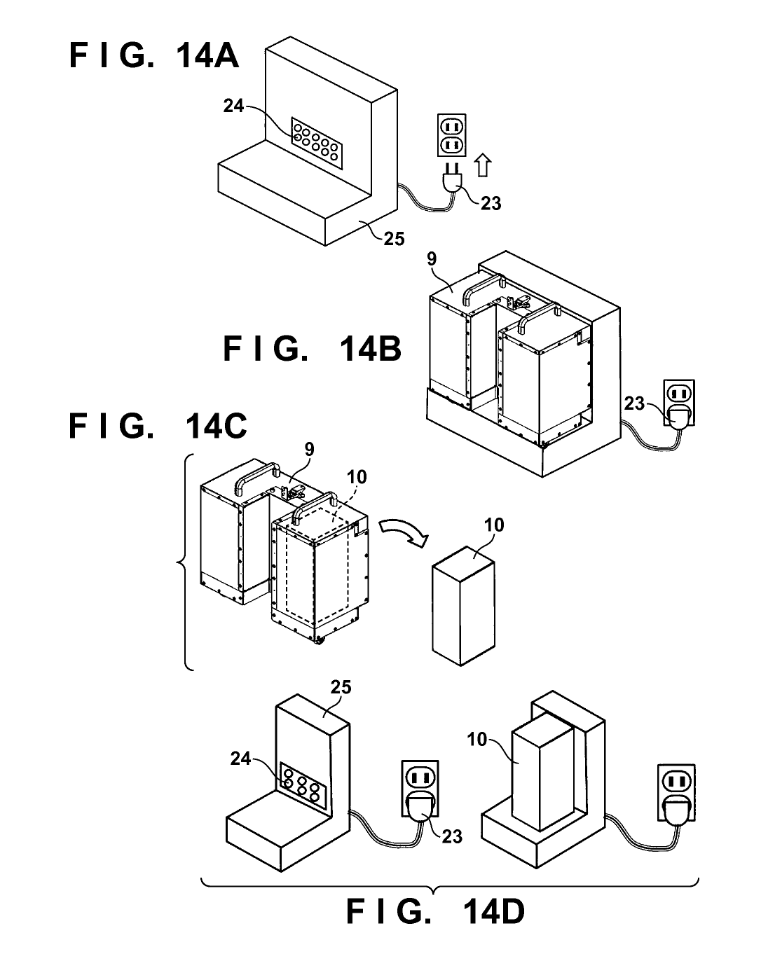

FIGS. 14A to 14D are views exemplifying an arrangement in which the battery is charged using a charging unit. The arrangement example described with reference to FIGS. 12A, 12B, 13A, and 13B requires work in which the operator conveys the power supply unit 9 or the battery 10 to a predetermined position and then connects the AC plug 23. To further reduce this work burden, the following structure may be adopted. A charging unit 25 is connectable to an AC power supply. The power supply unit 9 is configured to be detachable from the moving unit 8 (cart) held by a power supply holding member 32. By connecting the power supply unit 9 to the charging unit 25, the battery 10 of the power supply unit 9 is charged.

FIG. 14A shows the charging unit 25 including the AC plug 23 connectable to the AC power supply. The charging unit 25 has an energization terminal 24. Here, assume that the power supply unit 9 includes not the AC plug 23 but the energization terminal. FIG. 14B is a view showing a state in which the power supply unit 9 is mounted on the charging unit 25. When the power supply unit 9 is mounted on the charging unit 25, the energization terminal of the power supply unit 9 contacts the energization terminal 24 of the charging unit 25, electric power is supplied to the battery of the power supply unit 9, and the battery can be charged. By using the charging unit 25, the operator need not perform the AC plug connection work every time the battery is charged. The battery can be charged only by work of conveying the power supply unit 9 to the predetermined position where the charging unit 25 is installed.

The arrangement in FIGS. 14A and 14B represents an arrangement example in which the power supply unit 9 is detached from the moving unit 8 (cart) and conveyed to the charging unit 25 to charge the battery. The arrangement according to this embodiment is not limited to this example. It is also possible that, for example, the battery 10 is detached from the power supply unit 9 (FIG. 14C), and only the battery 10 is mounted on the charging unit 25 and charged (FIG. 14D). That is, the battery 10 is charged by connecting, to the charging unit 25, the battery 10 configured to be detachable from the power supply unit 9. This structure can further reduce the burden of the conveyance work on the operator. Note that the charging unit 25 can be mounted on the moving unit 8 (cart), and the radiation generator 1, the supporting unit 2, the power supply unit 9, and a radiation detector 7, grid 19, and information processing apparatus 20 which are accommodated in an accommodation unit 18 can be conveyed as an integral cart structure.

According to each of the above-described embodiments, the moving unit is configured to be detachable from the supporting unit, and capturing becomes possible regardless of whether the supporting unit is arranged on the moving unit or the supporting unit is separated from the moving unit.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-093167, filed Apr. 28, 2014, which is hereby incorporated by reference herein in its entirety.

What is claimed is:

1. A radiation generation apparatus comprising:
a radiation generator configured to generate radiation;
a supporting unit configured to support said radiation generator;
a power supply unit configured to supply electric power to said radiation generator; and
a moving unit configured to be movable with said power supply unit mounted on said moving unit,
wherein said moving unit is detachable from said supporting unit.

2. The apparatus according to claim 1, wherein said moving unit includes a holding member configured to detachably hold said supporting unit.

3. The apparatus according to claim 1, wherein said supporting unit includes a self-standing mechanism in which said supporting unit supports said radiation generator and stands by itself.

4. The apparatus according to claim 2, wherein when capturing is performed in a state in which the holding member holds said supporting unit on said moving unit, said radiation generator generates radiation on said moving unit.

5. The apparatus according to claim 4, wherein when capturing is performed in a state in which holding of the holding member is released and said supporting unit is separated from said moving unit, said radiation generator generates radiation in a state in which said supporting unit supports said radiation generator.

6. The apparatus according to claim 1, wherein said supporting unit includes:
a boom configured to support said radiation generator;
a column configured to support the boom; and
a supporting leg configured to support the column.

7. The apparatus according to claim 6, wherein the supporting leg includes:
stationary legs fixed to the column; and
movable legs configured to be able to be extended from the stationary legs.

8. The apparatus according to claim 7, wherein the movable legs are extended from a state in which the movable legs are folded with respect to the stationary legs.

9. The apparatus according to claim 7, wherein said supporting leg further includes sliding members configured to support the movable legs movably with respect to the stationary legs.

10. The apparatus according to claim 1, wherein a shielding member configured to shield the radiation is arranged on said moving unit.

11. The apparatus according to claim 1, further comprising an accommodation unit configured to be able to accommodate a detector configured to detect the radiation, a grid configured to remove a scattered ray of the radiation, and an information processing apparatus configured to process an image based on a detection result of the detector,
wherein said moving unit is movable with said accommodation unit further mounted on said moving unit.

12. The apparatus according to claim 2, wherein
said supporting unit includes an engaging member, and
the engaging member engages with the holding member of said moving unit and holds said supporting unit on said moving unit.

13. The apparatus according to claim 1, wherein said moving unit further includes a power supply holding member configured to detachably hold said power supply unit.

14. The apparatus according to claim 13, wherein building components of said power supply unit are arranged with a weight distribution equalized between left and right in a traveling direction of said moving unit.

15. The apparatus according to claim 14, wherein a housing of said power supply unit includes a concave portion formed in an outer shape of the housing along an arrangement of the building components.

16. The apparatus according to claim 14, wherein the building components include a battery configured to be detachable from said power supply unit.

17. The apparatus according to claim 13, further comprising a charging unit configured to be connectable to an AC power supply,
wherein said power supply unit is configured to be detachable from said moving unit held by the power supply holding member, and a battery of said power supply unit is charged by connecting said power supply unit to said charging unit.

18. The apparatus according to claim 17, wherein the battery is charged by connecting the charging unit and the battery configured to be detachable from said power supply unit.

19. A radiation generation apparatus comprising:
a radiation generator configured to generate radiation;
a supporting unit configured to support said radiation generator;
a power supply unit configured to supply electric power to said radiation generator; and
a moving unit including a plurality of wheels,
wherein said power supply unit is detachable from said moving unit, and said moving unit is detachable from said supporting unit.
